(19) United States
(12) Patent Application Publication
(10) Pub. No.: US 2024/0345211 A1
(43) Pub. Date: Oct. 17, 2024

(54) ELECTRONIC DEVICE AND CONTROL METHOD THEREOF

(71) Applicant: Wistron Corp., New Taipei City (TW)

(72) Inventors: Chuan Yen KAO, New Taipei City (TW); Yu Wen HUANG, New Taipei City (TW); Wei Rong TSENG, New Taipei City (TW); Yao Tsung CHANG, New Taipei City (TW); Yin Yu CHEN, New Taipei City (TW)

(21) Appl. No.: 18/331,386

(22) Filed: Jun. 8, 2023

(30) Foreign Application Priority Data
Apr. 13, 2023 (TW) 112113746

(51) Int. Cl. G01S 7/285; A61B 5/00; G01S 7/282; G01S 13/02

(52) U.S. Cl. CPC G01S 7/285; A61B 5/7221; G01S 7/282; G01S 13/02

(57) ABSTRACT

An electronic device and a control method thereof are provided. The electronic device includes a DSP (Digital Signal Processor). The DSP receives a digital signal. The digital signal includes a plurality of frames. The DSP divides the plurality of frames into a vital group and a non-vital group according to a criterion. The DSP compares a total number of frames of the vital group with a threshold value. In response to the total number of frames of the vital group being greater than the threshold value, the DSP may calculate signal strength of the vital group.

### CROSS REFERENCE TO RELATED APPLICATIONS

This application claims priority of Taiwan Patent Application No. 112113746 filed on Apr. 13, 2023, the entirety of which is incorporated by reference herein.

### BACKGROUND OF THE INVENTION

### Field of the Invention

The invention relates to an electronic device, and more particularly, to an electronic device and a control method thereof.

### Description of the Related Art

In a conventional design, detection devices for physiological signals tend to receive a lot of irrelevant noise. This not only wastes computing resources, but also makes it difficult to improve the quality of physiological signal analysis. Accordingly, there is a need for a novel solution to this problem in the prior art.

### BRIEF SUMMARY OF THE INVENTION

A preferred embodiment of the invention proposes an electronic device that includes a DSP (Digital Signal Processor). The DSP receives a digital signal. The digital signal includes a plurality of frames. The DSP divides the plurality of frames into a vital group and a non-vital group according to a criterion. The DSP compares a total number of frames of the vital group with a threshold value. In response to the total number of frames of the vital group being greater than the threshold value, the DSP calculates signal strength of the vital group.

A preferred embodiment of the invention proposes a control method used for an electronic device. The control method includes the following steps. A digital signal is received by the electronic device, wherein the digital signal includes a plurality of frames. The plurality of frames are divided into a vital group and a non-vital group according to a criterion. A total number of frames of the vital group are compared with a threshold value. In response to the total number of frames of the vital group being greater than the threshold value, signal strength of the vital group is calculated.

Based on the above descriptions, the electronic device and the control method of the invention may significantly improve its output accuracy according to practical measurements.

### BRIEF DESCRIPTION OF DRAWINGS

The invention may be more fully understood by reading the subsequent detailed description and examples with references made to the accompanying drawings, wherein:

FIG. 1 is a diagram of an electronic device according to an embodiment of the invention;

FIG. 2 is a diagram of a digital signal according to an embodiment of the invention;

FIG. 3 is a diagram of a frequency domain of a frame according to an embodiment of the invention;

FIG. 4 is a diagram of a digital signal according to an embodiment of the invention;

FIG. 5 is a diagram of an electronic device according to an embodiment of the invention;

FIG. 6 is a flowchart of operations of an electronic device according to an embodiment of the invention;

FIG. 7 is a flowchart of operations of an electronic device according to an embodiment of the invention; and

FIG. 8 is a flowchart of a control method according to an embodiment of the invention.

### DETAILED DESCRIPTION OF THE INVENTION

In order to illustrate the foregoing and other purposes, features and advantages of the invention, the embodiments and figures of the invention will be described in detail as follows.

The specification and claims do not intend to distinguish between components that differ in name but not function. In the following description and in the claims, the terms “include” and “comprise” are used in an open-ended fashion, and thus should be interpreted to mean “include, but not limited to . . . ”. Also, the term “couple” is intended to mean either an indirect or direct electrical connection. Accordingly, if one device is coupled to another device, that connection may be through a direct electrical connection, or through an indirect electrical connection via other devices and connections.

The following disclosure provides many different embodiments, or examples, for implementing different features of the subject matter provided. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

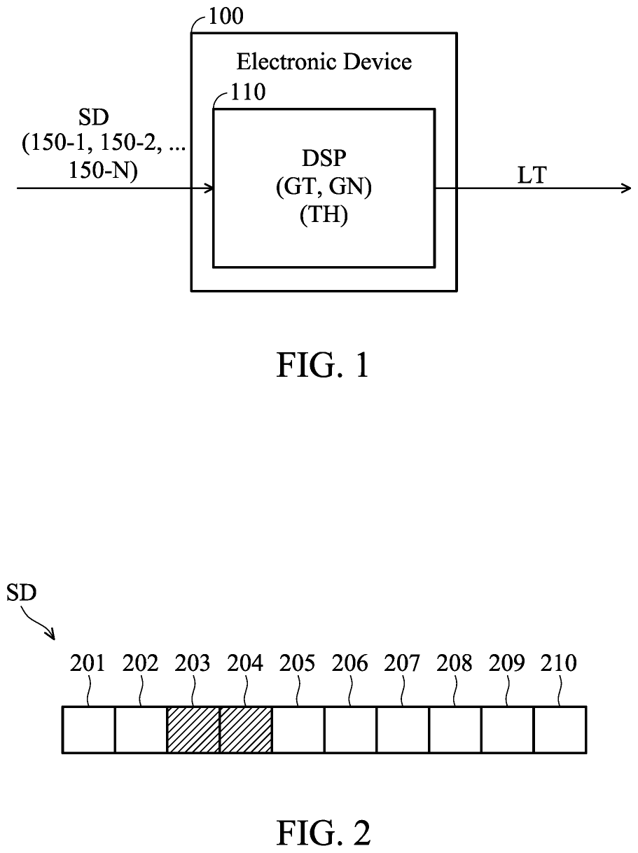

FIG. 1 is a diagram of an electronic device 100 according to an embodiment of the invention. The electronic device 100 may be applied to a radar device or a mobile device, such as a smart phone, a tablet computer, or a notebook computer, but it is not limited thereto. In the embodiment of FIG. 1, the electronic device 100 at least includes a DSP (Digital Signal Processor) 110. It should be understood that the electronic device 100 may further include other components, such as a housing, a transceiver, and/or a power supply module, although they are not displayed in FIG. 1.

The DSP 110 receives a digital signal SD. It should be noted that the DSP 110 sets the received digital signal SD into a plurality of frames in fixed segments, and then determines whether each frame belongs to a frame with a physiological signal (Vital Sign) or a frame without a physiological signal (Non-Vital Sign). The framework of the physiological signal received by the DSP 110 is filtered to reduce noise, through signal level adjustments and other pre-processing procedures to reduce the interference of signal frequencies, and then through the FFT (Fast Fourier Transform) algorithm to obtain the frequency domain response after the conversion. Next, the DSP 110 calculates whether the peak frequency of the frequency domain response is within the set reasonable physiological signal frequency. If the peak frequency is within the set reasonable physiological signal frequencies, it will be determined as a frame with a physiological signal, that is, the digital signal SD; otherwise, it will be determined as a frame without a physiological signal.

The digital signal SD includes a plurality of frames 150-1, 150-2, . . . , and 150-N, where “N” is a positive integer and may be greater than or equal to 10. Each frame may be considered as a time interval. For example, if the total duration of the digital signal SD is equal to 100 seconds, the frame 150-1 may represent a partial signal within a time interval from 0 to 10 seconds, the frame 150-2 may represent another partial signal within another time interval from 10 to 20 seconds, and so on. However, the invention is not limited thereto. In alternative embodiments, the total duration of the digital signal SD and the duration of each frame are adjustable according to different requirements.

The DSP 110 may divide the plurality of frames 150-1, 150-2, . . . , and 150-N into a vital group GT and a non-vital group GN according to a criterion. Such a criterion will be illustrated in detailed over the following embodiments. Next, the DSP 110 may compare the total number of frames of the vital group GT with a threshold value TH. For example, the threshold value TH may be equal to a half of the total number of frames of the digital signal SD, but it is not limited thereto. In response to the total number of frames of the vital group GT being greater than the threshold value TH, the DSP 110 may calculate signal strength LT of the vital group GT. On the contrary, in response to the total number of frames of the vital group GT being smaller than or equal to the threshold value TH, it may represent that meaningful frames are insufficient, and the DSP 110 may not generate any calculating results. With such a design, the DSP 110 of the proposed electronic device 100 may automatically exclude the frames affected by surrounding noise, so as to output the signal strength LT with relatively high accuracy.

The following embodiments will introduce different configurations and detail structural features of the electronic device 100. It should be understood that these figures and descriptions are merely exemplary, rather than limitations of the invention.

FIG. 2 is a diagram of the digital signal SD according to an embodiment of the invention. In the embodiment of FIG. 2, the digital signal SD includes a plurality of frames 201, 202, 203, 204, 205, 206, 207, 208, 209 and 210, which may be divided into a vital group GT and a non-vital group GN. For example, the vital group GT may include the frames 201, 202, 205, 206, 207, 208, 209 and 210, and the non-vital group GN may include the frames 203 and 204, but they are not limited thereto. It should be understood that the frames 201, 202, 205, 206, 207, 208, 209 and 210 of the vital group GT may be called as “vital frames”. The frames 203 and 204 of the non-vital group GN are affected by noise. Thus, the frames 203 and 204 may be called as “non-vital frames”. Furthermore, in the embodiment of FIG. 2, the aforementioned threshold value TH is equal to a half of the total number of frames of the digital signal SD (i.e., the number is 5), and therefore the total number of frames of the vital group GT (i.e., the number is 8) is greater than the aforementioned threshold value TH.

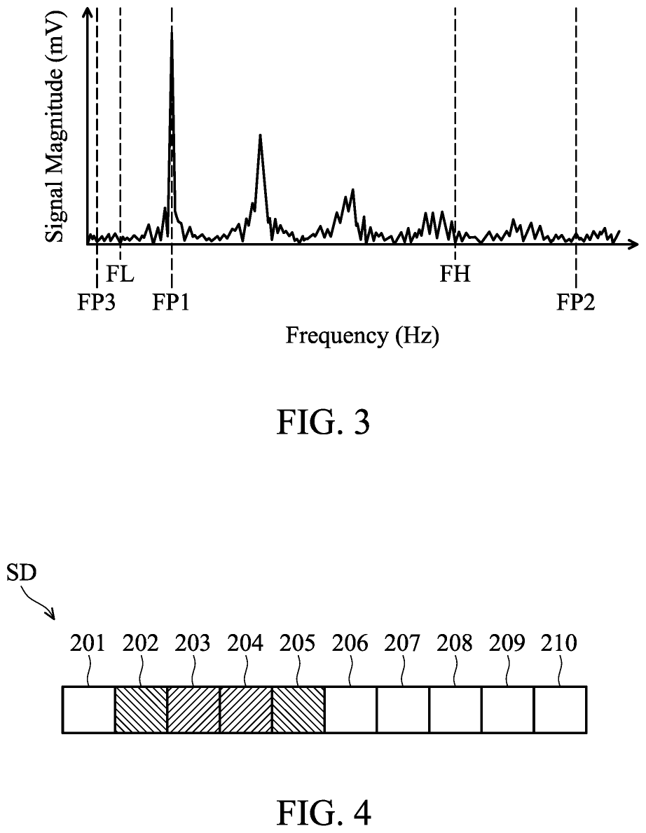

FIG. 3 is a diagram of a frequency domain of the frame 201 according to an embodiment of the invention, where the horizontal axis represents frequency (Hz), and the vertical axis represents signal magnitude (mV). It should be understood that the frame 201 is merely exemplary. As a matter of fact, the other frames may have similar characteristics. If the aforementioned DSP 110 performs an FFT on the frame 201, the relationship between its signal magnitude and frequency will be shown in FIG. 3. According to the measurement of FIG. 3, the frame 201 has a peak frequency FP1, which may correspond to the largest signal magnitude. It should be noted that the peak frequency of each frame of the vital group GT is between a down limit value FL and an up limit value FH. For example, the down limit value FL may be about 0.1 Hz, and the up limit value FH may be about 0.8 Hz, but they are not limited thereto. Conversely, the peak frequency of each of the frames 203 and 204 of the non-vital group GN (e.g., FP2 or FP3) may be lower than the down limit value FL, or may be higher than the up limit value FH. In other words, the criterion of the DSP 110 may be determined based on the frequency characteristics of each frame. However, the invention is not limited thereto. In other embodiments, the determination criterion of the DSP 110 is adjustable according to different requirements.

FIG. 4 is a diagram of the digital signal SD according to an embodiment of the invention. FIG. 4 is similar to FIG. 2. In the embodiment of FIG. 4, the non-vital group GN further includes the frames 202 and 205. It should be understood that the frames 202 and 205 of the non-vital group GN may be called as “adjacent frames”. Since the frames 202 and 205 are adjacent to the non-vital frames 203 and 204, they may be slightly affected by the noise. Thus, the frames 202 and 205 may also be excluded from the vital group GT. In other words, each of the adjacent frames 202 and 205 is adjacent to any one of the non-vital frames 203 and 204. According to practical measurements, if the adjacent frames 202 and 205 are removed from the vital group GT, the digital signal SD will not be interfered with so much, and the accuracy of the signal strength LT outputted by the DSP 110 will be further enhanced. Furthermore, in the embodiment of FIG. 4, the aforementioned threshold value TH is equal to a half of the total number of frames of the digital signal SD (i.e., the number is 5), and therefore the total number of frames of the vital group GT (i.e., the number is 6) is greater than the aforementioned threshold value TH.

In some embodiments, the digital signal SD includes 40 continuous frames F01 to F40, which will be described as the following Table I:

TABLE I
Frame and Magnitude of Digital Signal

| | Frame | | | | |
|---|---|---|---|---|---|
| | F01 | F02 | F03 | F04 | F05 |
| Magnitude | 12.6 | 12.3 | 11.7 | 12.7 | 11.6 |
| | Frame | | | | |
| | F06 | F07 | F08 | F09 | F10 |
| Magnitude | 13.4 | 13 | 13.8 | 13.5 | 12.4 |
| | Frame | | | | |
| | F11 | F12 | F13 | F14 | F15 |
| Magnitude | 13.7 | 13.2 | 12.6 | 11.9 | 97.3 |
| | Frame | | | | |
| | F16 | F17 | F18 | F19 | F20 |
| Magnitude | 97.3 | 21.1 | 11.7 | 13.3 | 17 |
| | Frame | | | | |
| | F21 | F22 | F23 | F24 | F25 |
| Magnitude | 21.4 | 23.7 | 38.6 | 29.5 | 23.3 |
| | Frame | | | | |
| | F26 | F27 | F28 | F29 | F30 |
| Magnitude | 13.9 | 10.7 | 14.3 | 21.8 | 25.4 |
| | Frame | | | | |
| | F31 | F32 | F33 | F34 | F35 |
| Magnitude | 31.6 | 26.2 | 21.5 | 19 | 17.1 |
| | Frame | | | | |
| | F36 | F37 | F38 | F39 | F40 |
| Magnitude | 15.4 | 16.7 | 18.8 | 21.6 | 97.3 |

For example, the frames F15, F16 and F40 may all be affected by noise, and they may be considered as “non-vital frames”. In addition, the frames F14, F17 and F39, which are adjacent to the aforementioned frames F15, F16 and F40, may be considered as “adjacent frames”. When the DSP 110 calculates the signal strength LT of the vital group GT, it may not take the non-vital frames F15, F16 and F40 and the adjacent frames F14, F17 and F39 of the non-vital group GN into account. Furthermore, in this embodiment, the aforementioned threshold value TH is equal to a half of the total number of frames of the digital signal SD (i.e., 20), and therefore the total number of frames of the vital group GT (i.e., 34) is greater than the aforementioned threshold value TH.

In some embodiments, the signal strength LT is equal to the average of the top 10% frames of the vital group GT, which may be calculated as follows. Initially, the total number of frames of the vital group GT may be divided by 10 (the remainder is not considered, that is, the remainder is unconditionally discarded). For example, the total number of frames of the vital group GT may be 34, and the quotient of dividing 34 by 10 may be 3. Then, the 10% frames with the largest amplitudes may be taken out from the vital group GT, and their average value may be calculated, which may be equivalent to the average of the top 10% frames. For example, the three frames with the largest amplitudes may be the frames F23, F31 and F24, respectively. The amplitude of the frame F23 may be 38.6. The amplitude of the frame F31 may be 31.6. The amplitude of the frame F24 may be 29.5. The average value of the above three frames is 33.23. Thus, the signal strength LT may be equal to 33.23.

In some embodiments, the signal strength LT is equal to the average of the bottom 10% frames of the vital group GT, which may be calculated as follows. Initially, the total number of frames of the vital group GT may be divided by 10 (the remainder is not considered, that is, the remainder is unconditionally discarded). For example, the total number of frames of the vital group GT may be 34, and the quotient of dividing 34 by 10 may be 3. Then, the 10% frames with the smallest amplitudes may be taken out from the vital group GT, and their average value may be calculated, which may be equivalent to the average of the bottom 10% frames. For example, the three frames with the smallest amplitudes may be the frames F27, F05 and F03 (or the frame F18), respectively. The amplitude of the frame F27 may be 10.7. The amplitude of the frame F05 may be 11.6. The amplitude of the frame F03 (or the frame F18) may be 11.7. The average value of the above three frames is 11.3. Thus, the signal strength LT may be equal to 11.3.

In some embodiments, the vital group GT merely excludes the non-vital frames, but does not exclude the adjacent frames. However, the invention is not limited thereto. In other embodiments, the signal strength LT is equal to an average value or a median of all of the frames of the vital group GT, and it is adjustable according to different requirements.

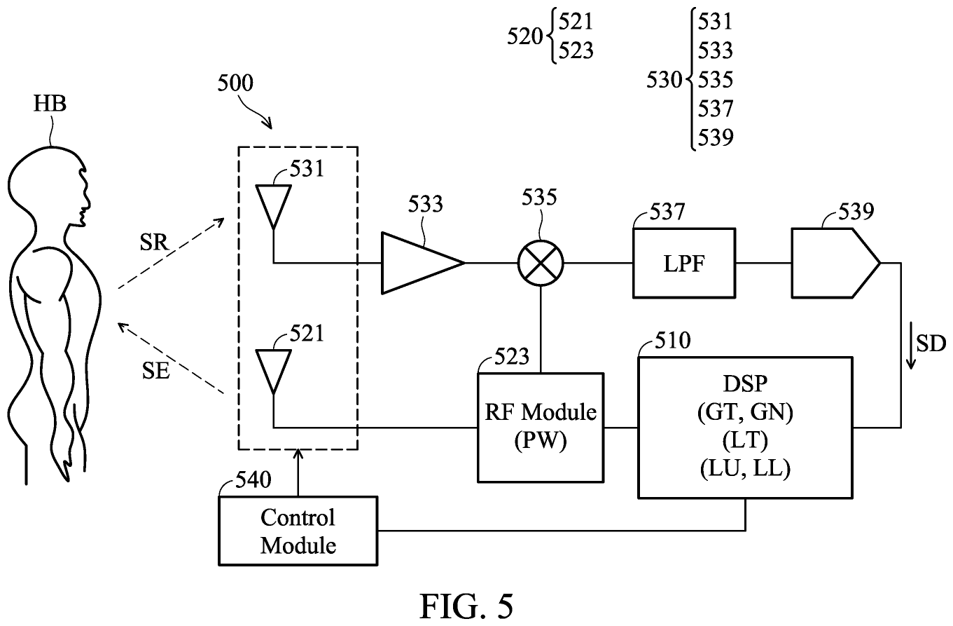

FIG. 5 is a diagram of an electronic device 500 according to an embodiment of the invention. In the embodiment of FIG. 5, the electronic device 500 includes a DSP 510, a transmitter module 520, a receiver module 530, and a control module 540. The operations of the DSP 510 have been described in the previous embodiments. Generally, the electronic device 500 is configured to detect an object. The object may be a human body HB. That is, the electronic device 500 is also configured to detect the physiological state of the human body HB.

The transmitter module 520 includes a transmission antenna 521 and an RF (Radio Frequency) module 523. The RF module 523 has transmission power PW. The transmission antenna 521 is coupled to the RF module 523. The transmission antenna 521 transmits a radar signal SE toward the human body HB. In response, the human body HB transmits a reflective signal SR back. The reflective signal SR may include the physiological information of the human body HB, such as a displacement, but it is not limited thereto.

The receiver module 530 includes a reception antenna 531, an LNA (Low Noise Amplifier) 533, a mixer 535, an LPF (Low-Pass Filter) 537, and an ADC (Analog-to-Digital Converter) 539. The above elements may be coupled in series, so as to form a reception path together. The reception antenna 531 receives the reflective signal SR from the human body HB. Next, the receiver module 530 generates a digital signal SD according to the reflective signal SR. Similarly, the DSP 510 divides a plurality of frames of the digital signal SD into a vital group GT and a non-vital group GN, and then calculates the signal strength LT of the vital group GT.

The shapes and types of the transmission antenna 521 and the reception antenna 531 are not limited in the invention. In some embodiments, any of the transmission antenna 521 and the reception antenna 531 is a monopole antenna, a dipole antenna, a patch antenna, a loop antenna, a PIFA (Planar Inverted F Antenna), or a chip antenna.

Specifically, the LNA 533 generates an amplified signal according to the input of the reception antenna 531. The RF module 523 provides an oscillation signal. The mixer 535 generates a mixed signal according to the amplified signal and the oscillation signal. The LPF 537 filters out high-frequency noise of the mixed signal, so as to generate a filtered signal. Next, the ADC 539 converts the filtered signal in analog form into the aforementioned digital signal SD.

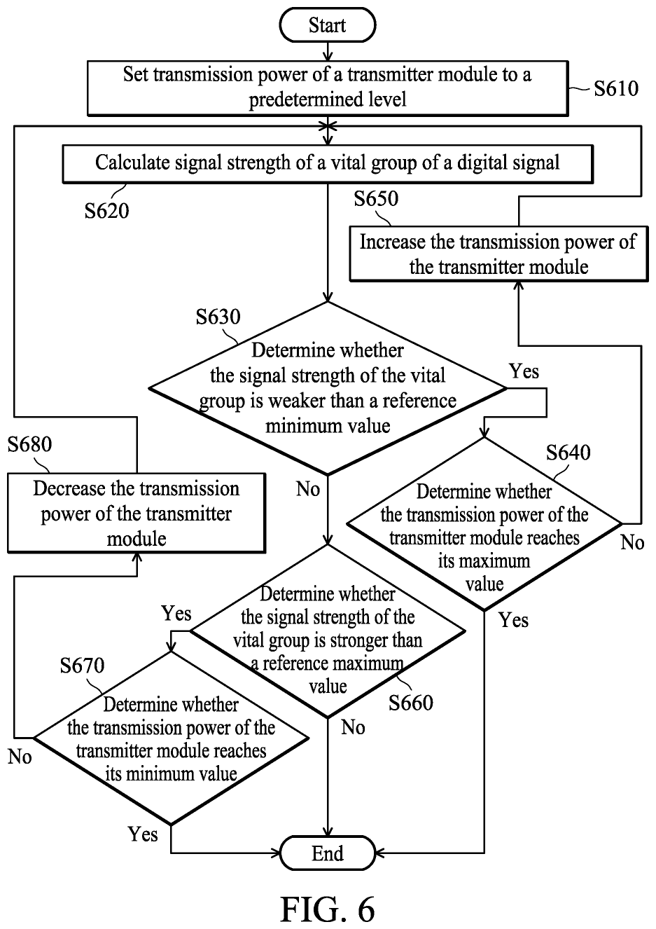

On the other hand, the control module 540 is coupled to the DSP 510. The control module 540 is controlled by the DSP 510, and is configured to selectively move the transmission antenna 521 and the reception antenna 531. For example, the control module 540 may include a mechanical arm and a driving motor, but it is not limited thereto. FIG. 6 is a flowchart of operations of the electronic device 500 according to an embodiment of the invention. To begin, in step S610, the DSP 510 sets the transmission power PW of the transmitter module 520 to a predetermined level. In step S620, the DSP 510 calculates the signal strength LT of the vital group GT of the digital signal SD. In step S630, the DSP 510 determines whether the signal strength LT of the vital group GT is weaker than a reference minimum value LL. If so, in step S640, the DSP 510 may determine whether the transmission power PW of the transmitter module 520 reaches its maximum value. If not, in step S650, the DSP 510 may increase the transmission power PW of the transmitter module 520. Conversely, if the signal strength LT of the vital group GT is stronger than or equal to the reference minimum value LL, in step S660, the DSP 510 may determine whether the signal strength LT of the vital group GT is stronger than a reference maximum value LU. If so, in step S670, the DSP 510 may determine whether the transmission power PW of the transmitter module 520 reaches its minimum value. If not, in step S680, the DSP 510 may decrease the transmission power PW of the transmitter module 520. It should be understood that these steps are not required to be performed in order. In some embodiments, if it is ensured that the predetermined level of the transmission power PW is between its minimum value and maximum value, the steps S640 and S670 may be omitted.

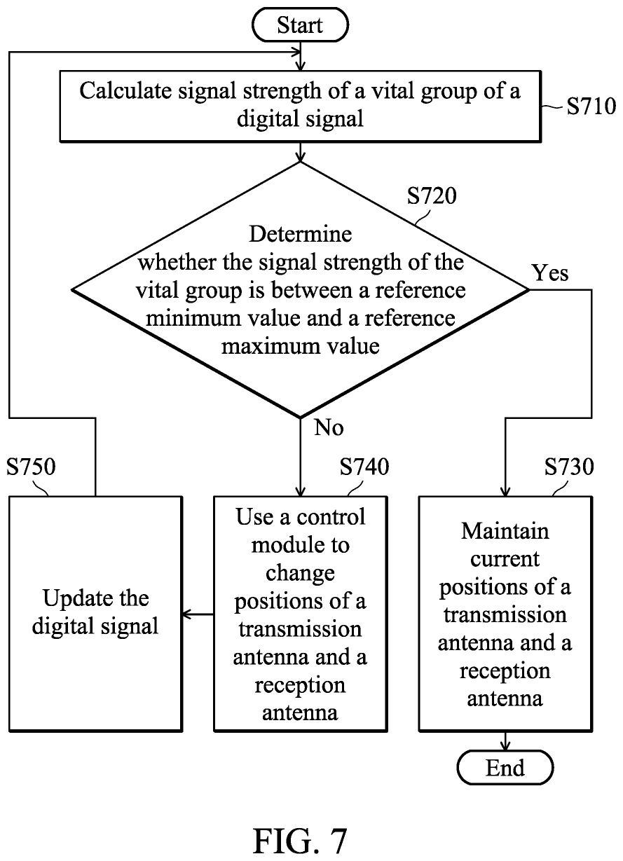

FIG. 7 is a flowchart of operations of the electronic device 500 according to an embodiment of the invention. To begin, in step S710, the DSP 510 calculates the signal strength LT of the vital group GT of the digital signal SD. In step S720, the DSP 510 determines whether the signal strength LT of the vital group GT is between the reference minimum value LL and the reference maximum value LU. If so, in step S730, the DSP 510 may take no action and maintain the current positions of the transmission antenna 521 and the reception antenna 531. Conversely, if not (i.e., the signal strength LT of the vital group GT is weaker than the reference minimum value LL, or is stronger than the reference maximum value LU), in step S740, the DSP 510 may use the control module 540 to change the positions of the transmission antenna 521 and the reception antenna 531. In step S750, the digital signal SD may be updated because of the movements of the transmission antenna 521 and the reception antenna 531, and the procedure will go back to step S710. At this time, the DSP 510 may recalculate the signal strength LT based on the updated digital signal SD and then perform the following process. It should be understood that these steps are not required to be performed in order. In some embodiments, the reference minimum value LL and the reference maximum value LU as mentioned above may be previously determined and stored in the DSP 510.

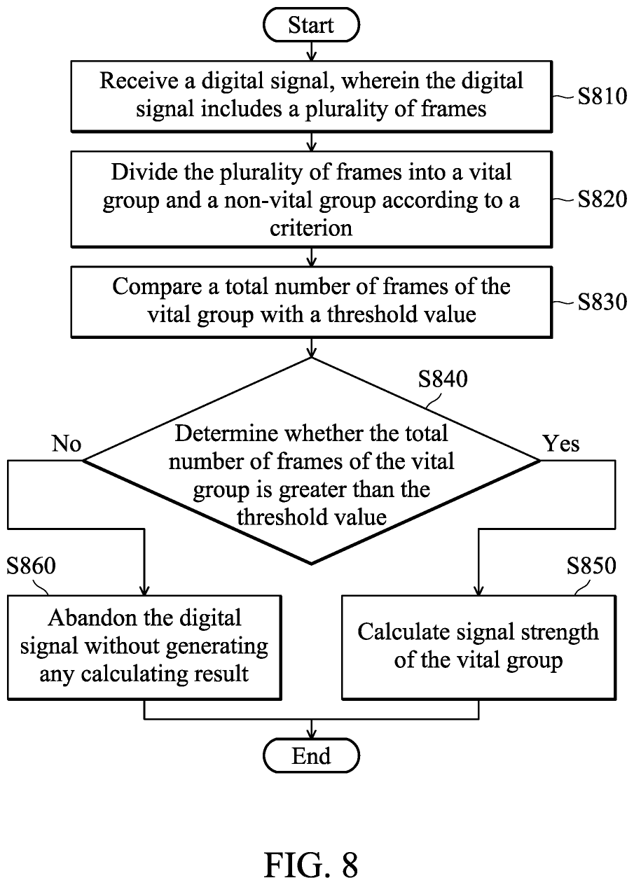

FIG. 8 is a flowchart of a control method according to an embodiment of the invention. To begin, in step S810, a digital signal is received. The digital signal includes a plurality of frames. In step S820, the plurality of frames are divided into a vital group and a non-vital group according to a criterion. In step S830, a total number of frames of the vital group is compared with a threshold value. In step S840, it is determined whether the total number of frames of the vital group is greater than the threshold value. If so, in step S850, the signal strength of the vital group may be calculated. If not, in step S860, the digital signal may be abandoned and no calculating result may be generated. It should be understood that these steps are not required to be performed in order, and every feature of the embodiments of FIGS. 1 to 7 may be applied to the control method of FIG. 8.

The invention proposed a novel electronic device and a novel control method. According to practical measurements, the electronic device using the aforementioned design may significantly improve its output accuracy. Therefore, the invention is suitable for application in a variety of detection systems.

Note that the above element parameters are not limitations of the invention. A designer may fine-tune these setting values according to different requirements. It should be understood that the electronic device and the control method of the invention are not limited to the configurations of FIGS. 1-8. The invention may include any one or more features of any one or more embodiments of FIGS. 1-8. In other words, not all of the features displayed in the figures should be implemented in the electronic device and the control method of the invention.

The method of the invention, or certain aspects or portions thereof, may take the form of program code (i.e., executable instructions) embodied in tangible media, such as floppy diskettes, CD-ROMS, hard drives, or any other machine-readable storage medium, wherein, when the program code is loaded into and executed by a machine such as a computer, the machine thereby becomes an apparatus for practicing the methods. The methods may also be embodied in the form of program code transmitted over some transmission medium, such as electrical wiring or cabling, through fiber optics, or via any other form of transmission, wherein, when the program code is received and loaded into and executed by a machine such as a computer, the machine becomes an apparatus for practicing the disclosed methods. When implemented on a general-purpose processor, the program code combines with the processor to provide a unique apparatus that operates analogously to application-specific logic circuits.

It will be apparent to those skilled in the art that various modifications and variations may be made in the invention. It is intended that the standard and examples be considered as exemplary only, with a true scope of the disclosed embodiments being indicated by the following claims and their equivalents.

What is claimed is:

1. An electronic device, comprising:
a DSP (Digital Signal Processor), receiving a digital signal, wherein the digital signal comprises a plurality of frames;
wherein the plurality of frames are divided into a vital group and a non-vital group by the DSP basing on a criterion;
wherein the DSP compares a total number of frames of the vital group with a threshold value;
wherein in response to the total number of frames of the vital group being greater than the threshold value, the DSP calculates a signal strength of the vital group.

2. The electronic device as claimed in claim 1, wherein a peak frequency of each of frames of the vital group is between a down limit value and an up limit value.

3. The electronic device as claimed in claim 2, wherein the non-vital group comprises a plurality of non-vital frames and a plurality of adjacent frames.

4. The electronic device as claimed in claim 3, wherein a peak frequency of each of the non-vital frames is lower than the down limit value or is higher than the up limit value.

5. The electronic device as claimed in claim 3, wherein each of the plurality of adjacent frames is adjacent to any one of the non-vital frames.

6. The electronic device as claimed in claim 1, further comprising:
a transmitter module, comprising a transmission antenna, and transmitting a radar signal toward an object;
a receiver module, comprising a reception antenna, and receiving a reflective signal from the object, wherein the receiver module generates the digital signal basing on the reflective signal; and
a control module, selectively moving the transmission antenna and the reception antenna.

7. The electronic device as claimed in claim 6, wherein the DSP compares the signal strength of the vital group with a reference minimum value and a reference maximum value, so as to adjust transmission power of the transmitter module.

8. The electronic device as claimed in claim 7, wherein in response to the signal strength of the vital group being weaker than the reference minimum value, the DSP increases the transmission power of the transmitter module, and wherein in response to the signal strength of the vital group being stronger than the reference maximum value, the DSP decreases the transmission power of the transmitter module.

9. The electronic device as claimed in claim 6, wherein the DSP compares the signal strength of the vital group with a reference minimum value and a reference maximum value, so as to adjust positions of the transmission antenna and the reception antenna.

10. The electronic device as claimed in claim 9, wherein in response to the signal strength of the vital group being weaker than the reference minimum value or being stronger than the reference maximum value, the DSP uses the control module to change the positions of the transmission antenna and the reception antenna, and wherein in response to the signal strength of the vital group being between the reference minimum value and the reference maximum value, the DSP maintains the positions of the transmission antenna and the reception antenna.

11. A control method used for an electronic device, and comprising the steps of:
receiving a digital signal by the electronic device, wherein the digital signal comprises a plurality of frames;
dividing the plurality of frames into a vital group and a non-vital group according to a criterion;
comparing a total number of frames of the vital group with a threshold value; and
in response to the total number of frames of the vital group being greater than the threshold value, calculating a signal strength of the vital group.

12. The control method as claimed in claim 11, wherein a peak frequency of each frame of the vital group is between a down limit value and an up limit value.

13. The control method as claimed in claim 12, wherein the non-vital group comprises a plurality of non-vital frames and a plurality of adjacent frames.

14. The control method as claimed in claim 13, wherein a peak frequency of each of the non-vital frames is lower than the down limit value or is higher than the up limit value.

15. The control method as claimed in claim 13, wherein each of the adjacent frames is adjacent to any one of the non-vital frames.

16. The control method as claimed in claim 11, further comprising:
transmitting a radar signal toward an object by the electronic device;
receiving a reflective signal from the object by the electronic device; and
generating the digital signal according to the reflective signal by the electronic device.

17. The control method as claimed in claim 16, further comprising:
comparing the signal strength of the vital group with a reference minimum value and a reference maximum value, so as to adjust transmission power of the electronic device.

18. The control method as claimed in claim 17, further comprising:
in response to the signal strength of the vital group being weaker than the reference minimum value, increasing the transmission power of the electronic device; and
in response to the signal strength of the vital group being stronger than the reference maximum value, decreasing the transmission power of the electronic device.

19. The control method as claimed in claim 16, further comprising:
comparing the signal strength of the vital group with a reference minimum value and a reference maximum value, so as to adjust positions of a transmission antenna and a reception antenna of the electronic device.

20. The control method as claimed in claim 19, further comprising:
in response to the signal strength of the vital group being weaker than the reference minimum value or being stronger than the reference maximum value, changing the positions of the transmission antenna and the reception antenna; and
in response to the signal strength of the vital group being between the reference minimum value and the reference maximum value, maintaining the positions of the transmission antenna and the reception antenna.
